(19) United States
(12) Patent Application Publication
(10) Pub. No.: US 2002/0173771 A1
(43) Pub. Date: Nov. 21, 2002

(54) Lesion restoration catheter for the colon and method for using same

(75) Inventor: Angel Dono, La Esperanza (SV)

Correspondence Address: Pablo Torres, La Tapachulteca II, 1500 South Vermont Avenue, Los Angeles, CA, 90006, US

(73) Assignee: La Tapachulteca II

(21) Appl. No.: 09/859,879

(22) Filed: May 17, 2001

(51) Int. Cl. A61M 29/00

(52) U.S. Cl. 604/540; 606/192; 604/96.01

(57) ABSTRACT

A device for removing waste from the small intestine so that the colon can be repaired. The method of using the device includes the steps of forming an opening in the colon, inserting the device through the opening in the colon, passing a portion of the device through the ileocecal valve, inflating a balloon associated with the device such that the balloon contacts an inner wall of the small intestine, whereby the balloon prevents waste in the small intestine from passing by the balloon, flowing waste through the device and outside of the body, and repairing the colon.

### FIELD OF THE INVENTION

The present invention relates to a device for allowing repair of the colon, and more particularly to a catheter that diverts waste from the small intestine to the outside of the body, thereby allowing the colon to be repaired.

### BACKGROUND OF THE INVENTION

When a person has some type of injury to the colon, whether it be from a gunshot, an infection, a blockage, or any other type of problem, the typical procedure is to perform a colostomy.

A colostomy is an alternative exit from the colon created to divert waste through a hole in the colon and through the wall of the abdomen. A colostomy is commonly performed by severing the colon to attach the end leading to the stomach to the skin through the wall of the abdomen. The end of the colon that leads to the rectum is closed off and becomes dormant. Usually a colostomy is performed for infection, blockage, or in rare instances, severe trauma of the colon. A colostomy is often performed so that an infection can be stopped and/or the affected colon tissues can heal.

In the past, a colostomy generally required that the bowel be extended out from an abdominal incision. A colostomy pouch, usually supported by a belt or girdle, was kept constantly in position to receive the fecal matter discharged during the colostomy. The colostomy pouch or bag is a removable, disposable bag that attaches to the exterior opening (stoma) to permit sanitary collection and disposal of bodily wastes. This arrangement is extremely objectionable due to the frequent escapage of foul odors and leakage of the fecal matter onto the body and wearing apparel. In one type of known colostomy procedure, instead of a belt or girdle, the colostomy bag is fixedly positioned against the outside of the abdominal wall with an adhesive. The disadvantage of using an adhesive is that the adhesive often causes irritation, which chafes or macerates the abdominal skin.

Generally, a colostomy is in place for a period of three to six months. The colostomy often entails complications that are resolved by performing additional surgeries. Even if there are no complications, at the end of the three to six month period, an additional surgery must be performed to remove the colostomy bag and stitch the two parts of the colon back together. These surgeries are major procedures and require recovery time for the patient. The hospital stay after the closing of a colostomy typically requires 4-5 days and results in an additional expense for the colostomy patient.

A colostomy is particularly important when the colon suffers a perforation, such as from a gunshot or stabbing. If the perforation is not repaired, the patient may die within a matter of hours. The flow of waste through the colon must be stopped to prevent the seepage of waste from the perforation. The flow of waste must also be stopped to allow the perforation to be repaired. Often in a severe perforation situation there is not enough time to perform a colostomy, which usually results in the patients death.

A long felt need exists for a method and device that limits the amount of time that a colon repair patient must spend in surgery, and that allows colon repair surgery to be quick and easy to perform.

### SUMMARY OF THE PREFERRED EMBODIMENTS

In accordance with a first aspect of the present invention there is provided a catheter for removal of waste from an intestine. The catheter is preferably used in conjunction with a cecostomy and controlled ileostomy to facilitate the lesion restoration of the colon. The catheter includes an outer tube having first and second ends, an inner tube having first and second ends, and which is concentric with the outer tube, a balloon associated with the first end of the inner tube, and an inlet member including an inlet opening associated with the second end of the inner tube.

The first and second ends of the inner tube extend outside of the first and second ends respectively of the outer tube. A channel is defined between the inner tube and the outer tube. In a preferred embodiment, the outer tube has an opening defined therein for allowing gas in the colon to pass into the outer tube and out of the second end (and, therefore, outside the body).

The device is intended to be used in a surgical procedure with an animal, preferably a human, body. As a result of the procedure, the second ends of the inner and outer tubes and the inlet member extend through an opening in the colon, the inner tube extends through the ileocecal valve, and the first end of the inner tube extends into the small intestine.

In accordance with another aspect of the present invention there is provided a method of repairing the colon. The method includes the steps of forming an opening in the colon, inserting the catheter through the opening in the colon, passing a portion of the device through the ileocecal valve, inflating a balloon associated with the device such that the balloon contacts an inner wall of the small intestine, whereby the balloon prevents waste in the small intestine from passing by the balloon, flowing waste through the catheter and outside of the body, and repairing the colon.

In accordance with another aspect of the present invention there is provided a method of removing waste from the small intestine. The method includes the steps of forming an opening in the colon, inserting the device through the opening in the colon, passing a portion of the catheter through the ileocecal valve, inflating a balloon associated with the device such that the balloon contacts an inner wall of the small intestine, whereby the balloon prevents waste in the small intestine from passing by the balloon, and flowing waste through the catheter and outside of the body. In a preferred embodiment, the method includes the step of passing gas from the colon through an opening in the outer tube and outside of the body.

In accordance with another aspect of the present invention there is provided a medical device that includes a pair of concentric tubes. Each tube has opposed ends, the outer tube has an opening between its ends and one end of one of the tubes is balloonable.

In accordance with another aspect of the present invention there is provided a medical device that includes a pair of concentric tubes including an inner tube and an outer tube each having opposed ends and a balloon disposed adjacent an end of one of the tubes and in flow communication with a fluid inlet member.

The fluid inlet member is disposed adjacent an end of one of the tubes opposite the balloon for facilitating inflation of the balloon. The end of the inner tube opposite the balloon is adapted for connection to a waste receiving member, such as a colostomy bag.

Other objects, features and advantages of the present invention will become apparent to those skilled in the art from the following detailed description. It is to be understood, however, that the detailed description and specific examples, while indicating preferred embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

The invention may be more readily understood by referring to the accompanying drawings in which

FIG. 1 is a elevational view showing a portion of a colon with the appendix extending therefrom and the small intestine connected to the colon.

FIG. 2 is a elevational view of a device for allowing repair of the colon in accordance with a preferred embodiment of the present invention. The device is shown in an environment wherein it has been inserted into the colon, through the ileocecal valve and into the small intestine of a human body.

FIG. 3 is a cross-sectional elevational view similar to FIG. 2, showing the inner components of the device for allowing repair of the colon.

FIG. 4 is a cross sectional end view taken along line 4--4 of FIG. 2.

Like numerals refer to like parts throughout the several views of the drawings.

### DETAILED DESCRIPTION OF THE PREFERRED EMBODIMENTS

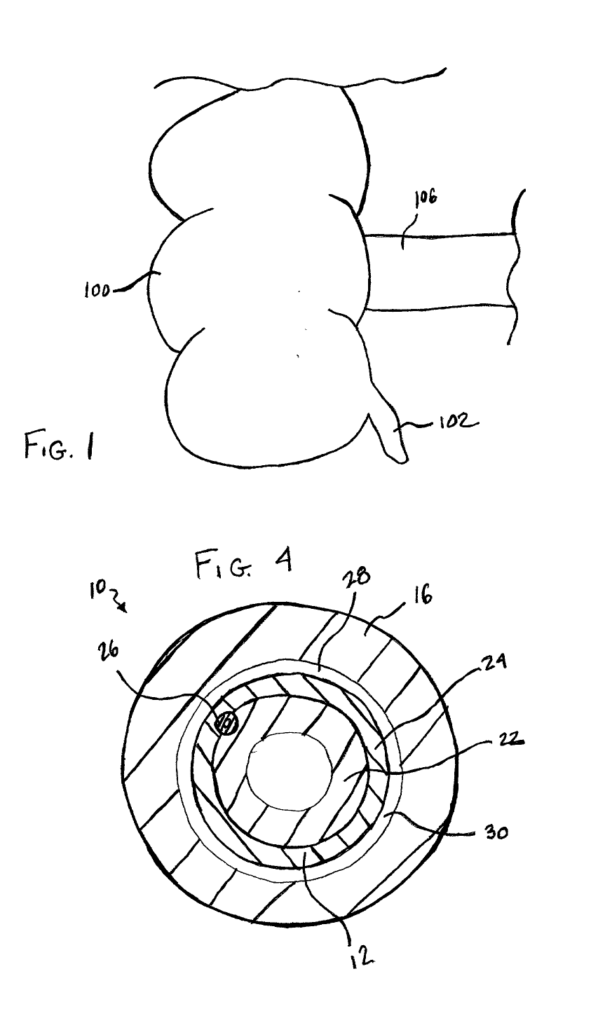

Referring generally to FIGS. 1-4, a first embodiment of a medical device or catheter 10 for allowing repair of the colon is shown. The device 10 generally includes an inner tube 12, a balloon 14, an outer tube 16 and a fluid inlet member 18. It will be appreciated that terms such as “below,” “forwardly,” “rearwardly,” “upwardly,” “downwardly” and “sideways” used herein are used merely for ease of description and refer to the orientation of the components as shown in the Figures.

It should be understood that any orientation of the device 10 described herein is within the scope of the present invention. As used herein, the term fluid means having particles that easily move and change their relative position without a separation of the mass and that easily yield to pressure. Any liquid or gas is a fluid within the scope of the present invention.

Referring to FIG. 1, the colon 100 is part of the large intestine, which is the tubelike organ that completes the process of digestion, receiving material from the small intestine 106. The large intestine has four parts: the cecum, the appendix 102, the colon 100, and the rectum. Once the products of digestion enter the cecum through the ileocecal valve 104 (which separates the large and small intestines), they move rapidly past the appendix, which juts out from the intestine near the cecum. The colon absorbs any remaining water and forms the stool, which is send to the rectum for elimination. The walls of the large intestine are muscular, and contract to move material along its length.

The small intestine 106 is the tubelike organ that receives the products of digestion from the stomach. The small intestine has three parts: the duodenum, the jejunum, and the ileum. The ileum ends with the ileocecal valve, which prevents food passed into the large intestine from traveling back into the small intestine 106. The walls of the small intestine 106 are muscular, and contract to move digested food along its length. The intestinal tube is lined with a mucus-like tissue that sends forth tiny, finger-like projections called villi. The villi increase the surface available for absorbing nutrients from digested food.

As discussed above, the appendix 102 is part of the large intestine. It is a small, finger-shaped pouch of intestinal tissue that juts out into the volume between the small intestine 106 and large intestine.

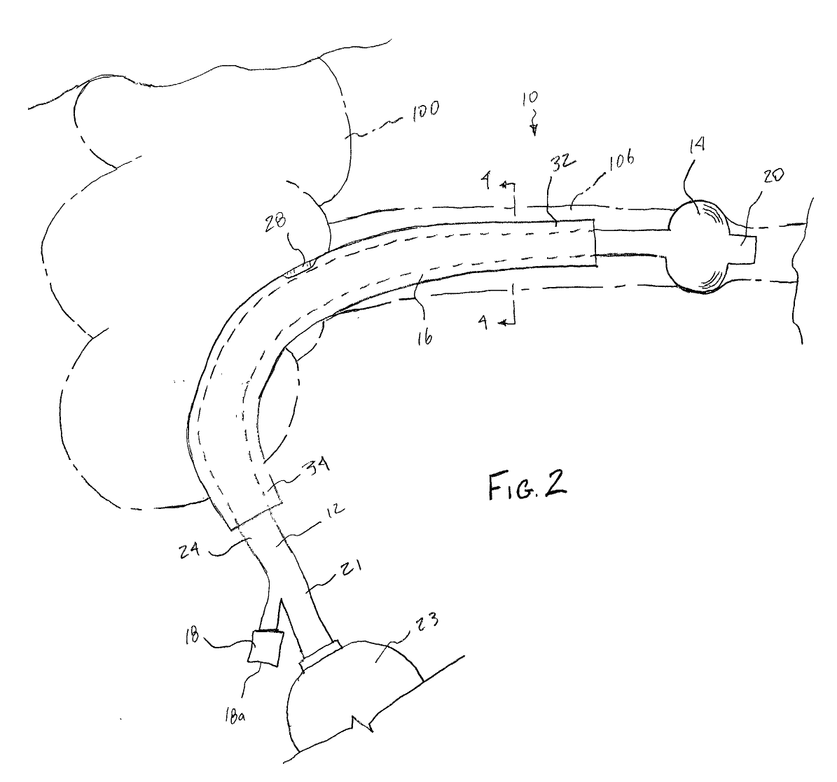

Referring to FIGS. 2-4, the inner tube 12 is hollow and is to be used for diverting the waste or fecal matter within the ileum to the outside of the body. The inner tube 12 has a first end 20 for receiving the waste, and an opposite second end 21 for disposing the waste outside of the body and preferably into a bag 23. The inner tube 12 is preferably formed of a plastic or rubber material that is safe to use in the body. In a preferred embodiment, the inner tube is formed of two layers, a first or inner layer 22 that is formed of a rigid plastic, and a second or outer layer 24 that is formed of a more pliable plastic or rubber. It will be understood that the inner tube 12 can be formed of a single layer or of multiple layers.

The balloon 14 is formed of an elastic plastic or rubber material and is preferably formed in the second layer 24 of the inner tube, adjacent the first end 20 of the tube. In another embodiment, the balloon 14 can be attached to the outside surface of the inner tube 12. When the device 10 is positioned within the small intestine and the balloon 14 is inflated, the balloon 14 fully occupies the space between the device 10 and the inside surface of the small intestine. The inflated balloon 14 completely blocks the waste flowing through the small intestine and fully diverts the waste into the first end 20 of the inner tube 12. In a preferred embodiment of the invention, the balloon 14 has a volume of 35 cubic centimeters when fully inflated.

The balloon 14 is filled with air or liquid by a narrow tubular member 26 that preferably extends between first and second layers 22, 24 of inner tube 12 and terminates in an opening 18a in fluid inlet member 18. The tubular member 26 extends into the inside of balloon 14 for inflation thereof. In another embodiment, the tubular member 26 can be attached to the inside surface of inner tube 12. In yet another embodiment, the tubular member 26 can be formed inside the wall of inner tube 12, such as by molding.

Devices for filling balloon 14 are well known in the art. For example, the device may include a one way valve that is attached to an inlet end of the tubular member 26 at fluid inlet member 18, or a second tubular member may be connected to fluid inlet member 18 and the one way valve connected to an inlet end of the second tubular member. It will be understood that any device that is in fluid communication with the tubular member 26 and is capable of inflating balloon 14 with fluid is within the scope of the present invention.

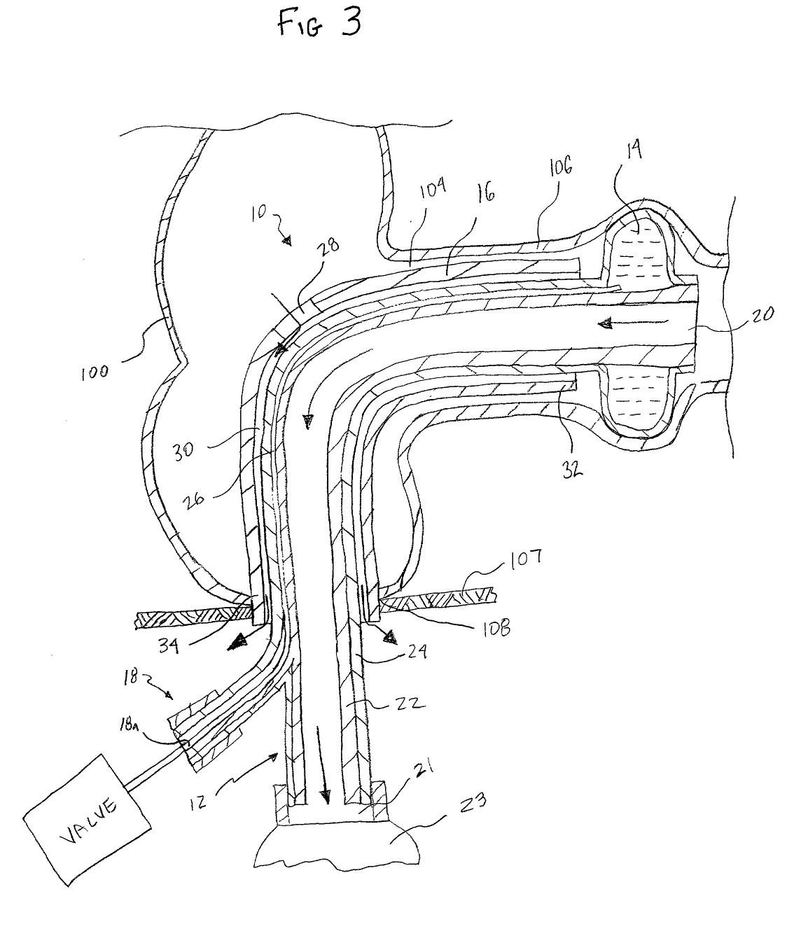

The inner tube 12 is concentric with the outer tube 16. The outer tube 16 is preferably formed of a plastic or rubber material that is safe to use in the body. In a preferred embodiment of the invention, the outer tube has a rigid, L-shaped form and is approximately 27 to 30 cm in length. As shown in FIG. 3, an opening 28 is formed in the outer tube 16 in a location such that it will be positioned in the colon 100 after placement of the device 10 within the body. As will be described below, gas in the colon will exit the body through opening 28. A space 30 is defined between the inner and outer tubes 12, 16 to allow gas to pass through the outer tube 16. The outer tube 16 is preferably not as long as the inner tube 12. The portion of the inner tube 12 that includes the balloon 14 extends outside the outer tube 16 so that the balloon 14 has room to inflate. The outer tube 16 includes a first end 32 and a second/gas disposal end 34. In a preferred embodiment, the inner tube 12 is secured within the outer tube 16 by glue or other adhesive. However, it will be understood that the inner tube 12 may be secured inside the outer tube 16 by any known method. For example, the concentric tubes may be formed integrally with another, such as by molding.

To begin the procedure of positioning the device 10 within the patient, a small incision is made in the lower right side of the abdomen 107 at a location to afford access to the appendix 102. The appendix 102 is then removed. The removal of the appendix is a common procedure and for sake of efficiency will not be described here. After removal of the appendix 102, an incision 108 is made in the colon 100 where the appendix 102 was formerly located. In an alternative embodiment, an incision can be made adjacent the appendix 102, without having to remove the appendix.

The first end 20 of the inner tube 12 is inserted into the incision in the abdomen and the incision 108 in the colon 100. The first end 20 is then passed through the ileocecal valve 104 and into the ileum of the small intestine 106. In a preferred embodiment of the invention, the device is inserted in the ileocecal valve 104 approximately 15 cm. The first end 34 of the outer tube 16 is also passed through the incision 108 and the ileocecal valve 104. Because the outer tube 16 is used to evacuate gas from the colon 100, the outer tube 16 may be short enough that it does not pass through the ileocecal valve 104. In other words, it is only important that the inner tube 12 passes through the ileocecal valve and into the ileum. Due to the musculature of the colon 100, the incision 108 remains tight around the outer tube 16, as shown in FIG. 3.

After the device 10 has been positioned appropriately, a device for inflating the balloon 14 is placed in fluid communication with the tubular member 26 and the balloon 14 is inflated to a point where the small intestine 106 is sealed so that no waste can pass between the wall of the small intestine 106 and the balloon 14. After the balloon 14 has been inflated, because the small intestine 106 is blocked, the waste in the small intestine 106 enters the first end 20 of the inner tube 12 and passes therethrough to the second end 21 and exits into the bag 23 or other disposal apparatus. Colostomy bags are well known in the art. Therefore the type of bag 23 used for the present invention is not limiting.

Because the waste from the small intestine 106 is diverted outside of the body and not into the colon 100, the colon 100 remains empty and can then be repaired. After the surgery for inserting the device 10 and repairing the colon 100, the colon must then heal. The healing process varies from patient to patient and can take anywhere from about fifteen days to six weeks. The amount of time the device 10 is left in place is not a limitation on the present invention.

After the colon 100 has been allowed to heal, the device 10 can then be removed from the patient's body. To remove the device 10, the balloon 14 is deflated, and the device 10 is pulled back through the ileocecal valve 104, the incision 108 in the colon (where the appendix was) and the incision in the abdominal wall. The musculature of the sphincter of the ileocecal valve 104 regains its tone when the device 10 is withdrawn, regardless of the length of time that the device was left therein. Accordingly, the requirement of suturing of the colostomy is eliminated.

The device of the present invention and the method of placement of the device allow the restoration of lesions in the colon in a fast, convenient, and cost-effective manner. A typical colostomy usually requires the patient to wear the colostomy bag and an uncomfortable girdle for three to six months, after which a second surgery is required to reattach the sections of the colon and remove the colostomy bag. The present invention requires a single surgery, avoids the above-mentioned inconveniences during the several months of convalescence that is required for known colostomy procedures, and saves the patient the cost and inconvenience of a second surgery to either correct the complications of the colostomy or to close the colostomy. Moreover, the patient does not need to purchase accessories to ensure hygienic conditions on the patient's body.

The embodiments of the present invention recited herein are intended to be merely exemplary and those skilled in the art will be able to make numerous modifications to them without departing from the spirit of the present invention. All such modifications are intended to be within the scope of the present invention as defined by the claims appended hereto.

What is claimed is:

1. A device for removal of waste from an intestine, said device comprising:
a) an outer tube having first and second ends,
b) an inner tube having first and second ends, and concentric with said outer tube, wherein said first and second ends of said inner tube extend outside of said first and second ends respectively of said outer tube, wherein a space is defined between said inner tube and said outer tube,
c) a balloon associated with said first end of said inner tube, and
d) an inlet member including an inlet opening associated with said second end of said inner tube.

2. The device of claim 1 wherein said outer tube has an opening defined therein.

3. The device of claim 1 wherein said balloon is formed as a unit with said inner tube.

4. The device of claim 1 wherein said inner tube includes an inner layer and an outer layer, and wherein said outer layer forms a part of said balloon.

5. The device of claim 1 wherein a narrow tubular member extends from said inlet member to said balloon and communicates said inlet opening with said balloon.

6. The device of claim 1 wherein an outside surface of said inner tube is secured to an inside surface of said outer tube with an adhesive.

7. The device of claim 5 wherein said narrow tubular member extends between said first and second layers of said inner tube.

8. The device of claim 1 in combination with an animal body, wherein said second ends of said inner and outer tubes and said inlet member extend through an opening in the colon, wherein said inner tube extends through the ileocecal valve, and wherein said first end of said inner tube extends into the small intestine.

9. The combination of claim 8 wherein said outer tube also extends through the ileocecal valve and into the small intestine.

10. The combination of claim 8 wherein said outer tube has an opening defined therein, and wherein said opening is positioned in the colon.

11. A method of repairing the colon within an animal body, the method comprising the steps of
(a) forming an opening in the colon,
(b) inserting a device through said opening in the colon,
(c) passing a portion of said device through the ileocecal valve,
(d) inflating a balloon associated with said device, wherein said balloon contacts an inner wall of the small intestine, whereby inflation of said balloon prevents waste in the small intestine from passing by said balloon,
(e) flowing waste through said device and outside of the body, and
(f) repairing the colon.

12. The method of claim 11 wherein said opening in the colon is formed by removing the appendix.

13. The method of claim 11 wherein said device includes an outer tube and an inner tube concentric with said outer tube.

14. The method of claim 13 wherein said outer tube has an opening defined therein that is positioned in the colon after performance of step (c).

15. The method of claim 14 further comprising the step of passing gas from the colon through said opening in said outer tube and outside of the body.

16. The method of claim 11 wherein said inner tube is passed through the ileocecal valve in step (c).

17. The method of claim 13 wherein said inner and outer tubes each have first and second ends, wherein said first end of said inner tube extends outside of said first end of said outer tube, and wherein said balloon is associated with said first end of said inner tube.

18. The method of claim 17 wherein said second end of said inner tube extends outside of said second end of said outer tube, and wherein said device includes an inlet member communicated with said balloon associated with said second end of said inner tube.

19. The method of claim 18 wherein step (d) includes flowing a fluid through said inlet member and into said balloon.

20. The method of claim 11 further comprising the steps of waiting an amount of time to allow the colon to heal, and removing said device from said body by pulling the device back through the ileocecal valve, the opening in the colon and outside of the body.

21. A method of removing waste from the small intestine, the method comprising the steps of
(a) forming an opening in the colon,
(b) inserting a device through said opening in the colon,
(c) passing a portion of the device through an ileocecal valve,
(d) inflating a balloon associated with said device, wherein said balloon contacts an inner wall of a small intestine, whereby said balloon prevents waste in said small intestine from passing by said balloon, and
(e) flowing waste through said device and outside of the body.

22. A medical device comprising a pair of concentric tubes, each tube having opposed ends, the outer tube having an opening between the ends of the outer tube, one end of one of the tubes being balloonable.

23. A medical device comprising:
a) a pair of concentric tubes including an inner tube and an outer tube each having opposed ends,
b) a balloon disposed adjacent an end of one of the tubes and in flow communication with a fluid inlet member, said fluid inlet member being disposed adjacent an end of one of the tubes opposite the balloon for facilitating inflation of the balloon, and
c) the end of the inner tube opposite the balloon being adapted for connection to a waste receiving member.
